(19) United States
(12) Patent Application Publication
(10) Pub. No.: US 2019/0119650 A1
(43) Pub. Date: Apr. 25, 2019

(54) EVALUATION METHOD FOR DIFFERENTIATION STATE OF CELLS

(71) Applicants: SHIMADZU CORPORATION, Kyoto-shi (JP); TOKYO ELECTRON LIMITED, Minato-ku (JP)

(72) Inventors: Takashi SUZUKI, Kyoto-shi (JP); Daisuke HIRAMARU, Kyoto-shi (JP); Masatoshi TAKAHASHI, Kyoto-shi (JP); Kentaro TOMITA, Minato-ku (JP); Kunitada HATABAYASHI, Minato-ku (JP); Kenichi KAGAWA, Minato-ku (JP); Shigenori OZAKI, Nirasaki City (JP)

(73) Assignees: SHIMADZU CORPORATION, Kyoto-shi (JP); TOKYO ELECTRON LIMITED, Minato-ku (JP)

(21) Appl. No.: 15/769,784

(22) PCT Filed: Mar. 16, 2016
(86) PCT No.: PCT/JP2016/058303

§ 371 Date: Jul. 31, 2018

(30) Foreign Application Priority Data
Oct. 23, 2015 (JP) PCT/JP2015/080003

(51) Int. Cl. C12N 5/074; G01N 33/50

(52) U.S. Cl. CPC C12N 5/0696; C12N 5/0603; G01N 33/5073

(57) ABSTRACT

The present invention is a method in which stem cells for which a differentiation state is unknown or cells that are differentiation-induced from the stem cells are use as test cells, and a differentiation state of the test cells is evaluated based on an abundance of a predetermined indicator substance in a culture supernatant of the test cells. The indicator substance is at least one compound selected from a group that consists of ornithine, 2-aminoadipic acid, deoxycytidine, glutamic acid, tryptophan, asparagine, alanine, cystine, hypoxanthine, uridine, aspartic acid, arginine, 2-hydroxybutyric acid, 2-hydroxyisovaleric acid, 3-hydroxyisovaleric acid, urea, 4-hydroxybenzoic acid, 4-aminobenzoic acid, ribonic acid, kynurenine, crystathionine, threonic acid, pyruvic acid, putrescine, ascorbic acid, riboflavin, serine, cysteine, orotic acid, and citric acid. According to an embodiment of the present invention, a differentiation state is evaluated based on abundances of two or more kinds of indicator substances.

### TECHNICAL FIELD

The present invention relates to a method for evaluating a differentiation state of cells.

### TECHNICAL BACKGROUND

Conventionally, in order to evaluate a differentiation state of cells, a method in which immunostaining is used (for example, see Patent Document 1) and a method in which an expression level of a marker gene is quantified (for example, see Patent Document 2) are widely used.

In the method in which immunostaining is used, first, cells, for example, pluripotent stem cells, which are to be evaluated, are immobilized using paraformaldehyde or the like, and then, are subjected an antigen-antibody reaction. Here, SSEA-4 or TRA1-60 is widely used as an antibody for determining whether or not the pluripotent stem cells are in an undifferentiated state (for example, see Patent Document 1). Subsequently, a secondary antibody that binds to the antibody is added to the cells, and then a fluorescent label or the like attached to the secondary antibody in advance is detected. As a result, whether or not an antigen to the antibody exists on the cells, that is, whether or not the cells are in an undifferentiated state can be evaluated.

Further, in the method based on quantification of an expression level of a marker gene, for example, mRNA is extracted from the pluripotent stem cells and is converted to cDNA using reverse transcriptase, and then, the marker gene is amplified by a PCR (polymerase chain reaction). In this case, NANOG or POU5F1 (October 3/4) is widely used as a marker gene for evaluating an undifferentiated property of the pluripotent stem cells (for example, see Non-Patent Document 1). By detecting a product of the PCR using electrophoresis or a real time PCR device, the expression level of the marker gene in the cells is confirmed, and, from the result, whether or not the cells are in an undifferentiated state is evaluated.

### RELATED ART

### Patent Documents

Patent Literature 1: Japanese Patent Laid-Open Publication No. 2004-313184.

Patent Document 2: Japanese Patent Laid-Open Publication No. 2006-042663.

### Non-Patent Document

Non-Patent Document 1: Nature Biotechnology, 2007, Volume 25, pages 803-816.

### SUMMARY OF THE INVENTION

### Problems to Be Solved By the Invention

However, in all of the above-described conventional evaluation methods, it is necessary to perform an invasive treatment to the cells. Therefore, after evaluating a differentiation state, cells subjected to the evaluation could not be used for other purposes, for example, as a cell source for regenerative medicine. Further, for the same sample (that is, cells in the same culture dish), it was not possible to evaluate change with time. In order to evaluate temporal change of a differentiation state, complicated work, such as culturing multiple culture dishes in parallel, was necessary.

The present invention is accomplished in view of the above problem and is intended to provide a method for noninvasively evaluating a differentiation state of cells.

### Means for Solving the Problems

As a result of intensive studies, the present inventors found that an abundance of a specific indicator substance in a culture supernatant varies depending on a differentiation state of cells, and thus accomplished the present invention.

In a cell differentiation state evaluation method according to the present invention, stem cells for which a differentiation state is unknown, or cells that are differentiation-induced from the stem cells are used as test cells, and a differentiation state of the test cells is evaluated based on an abundance of a predetermined indicator substance in a culture supernatant of the test cells. That is, the cell differentiation state evaluation method according to the present invention includes a step of measuring an abundance of an indicator substance in a culture supernatant of test cells, and a step of evaluating a differentiation state of the test cells based on the abundance of the indicator substance.

The indicator substance is at least one compound selected from a group that consists of ornithine, 2-aminoadipic acid, deoxycytidine, glutamic acid, tryptophan, asparagine, alanine, cystine, hypoxanthine, uridine, aspartic acid, arginine, 2-hydroxybutyric acid, 2-hydroxyisovaleric acid, 3-hydroxyisovaleric acid, urea, 4-hydroxybenzoic acid, 4-aminobenzoic acid, ribonic acid, kynurenine, cystathionine, threonic acid, pyruvic acid, putrescine, ascorbic acid, riboflavin, serine, cysteine, orotic acid, and citric acid.

In the cell differentiation state evaluation method according to the present invention, for example, a differentiation state of test cells can be evaluated by comparing an abundance of an indicator substance in a culture supernatant of the test cells with an abundance of the indicator substance in a culture supernatant of control cells for which a differentiation state is known.

For example, for an indicator substance such as arginine, tryptophan, or 4-aminobenzoic acid, an abundance in a culture supernatant of differentiated cells tends to be larger than an abundance in a culture supernatant of undifferentiated cells. Therefore, in a case where cells that are clearly undifferentiated are used as control cells, when a ratio of an abundance of an indicator substance such as arginine, tryptophan or 4-aminobenzoic acid in a culture supernatant of test cells to an abundance of the indicator substance in a culture supernatant of the control cells is equal to or greater than a predetermined threshold, it can be determined that the test cells are in a differentiated state.

For example, for an indicator substance such as ornithine, alanine, aspartic acid, 2-hydroxybutyric acid, 2-hydroxyisovaleric acid, urea, or 4-hydroxybenzoic acid, an abundance in a culture supernatant of differentiated cells tends to be smaller than an abundance in a culture supernatant of undifferentiated cells. Therefore, in the case where cells that are clearly undifferentiated are used as control cells, when a ratio of an abundance of an indicator substance such as ornithine, alanine, aspartic acid, 2-hydroxybutyric acid, 2hydroxyisovaleric acid, urea, or 4-hydroxybenzoic acid in a culture supernatant of test cells to an abundance of the indicator substance in a culture supernatant of the control cells is equal to or less than a predetermined threshold, it can be determined that the test cells are in a differentiated state.

According to an embodiment of the present invention, a differentiation state is evaluated based on abundances of two or more kinds of the above-described indicator substances. For example, the differentiation state is evaluated by comparing, for each of two or more kinds of indicator substances, an abundance of an indicator substance in a culture supernatant of the test cells with an abundance of the indicator substance in a culture supernatant of the control cells. It is also possible to obtain, for each of two or more kinds of indicator substances, a ratio of an abundance on an indicator substance in a culture supernatant of the test cells to an abundance of the indicator substance in a culture supernatant of the control cells, and to evaluate the differentiation state based on whether or not these ratios are equal to or greater than a predetermined threshold. As the target cells, for example, cells that are clearly undifferentiated are used.

As two or more kinds of indicator substances, for example, a precursor and a metabolic product in an anteroposterior relation of a metabolic pathway can be selected, and the differentiation state can be evaluated based on a ratio of an abundance of the precursor to the metabolic product in a culture supernatant of the test cells. Examples of a combination of a precursor and a metabolic product include a combination of tryptophan and kynurenine, a combination of ornithine and putrescine, a combination of arginine and ornithine, and the like.

As two or more kinds of indicator substances, for example, a combination including kynurenine, 2-aminoadipic acid, ornithine and deoxycytidine can be adopted. Two or more kinds of indicator substances may further include tryptophan, cystathionine, hypoxanthine, and uridine.

By evaluating the differentiation state based on abundances of two or more kinds of indicator substances, the differentiation state of the test cells can be evaluated with higher accuracy. Further, by evaluating the differentiation state based on abundances of two or more kinds of indicator substances, in addition to evaluation of whether or not the test cells are in a differentiated state or in an undifferentiated state, it is also possible to evaluate whether a differentiation direction of the test cells that have been evaluated as being in a differentiated state is endoderm, mesoderm, or ectoderm.

For example, by analyzing changes with culture time of abundances of two or more kinds of indicator substances selected from a group consisting of kynurenine, 2-aminoadipic acid and deoxycytidine in a culture supernatant of the test cells, whether or not the test cells are in a differentiated state or in an undifferentiated state can be evaluated, and whether a differentiation direction of the test cells that have been evaluated as being in a differentiated state is endoderm, mesoderm, or ectoderm can be evaluated.

By subjecting abundances of two or more kinds of indicator substances selected from a group consisting of kynurenine, ornithine, 2-aminoadipic acid, deoxycytidine, tryptophan, cystathionine, hypoxanthine, and uridine in a culture supernatant of the test cells to multivariate analysis, whether or not the test cells are in a differentiated state or in an undifferentiated state can be evaluated, and whether a differentiation direction of the test cells that have been evaluated as being in a differentiated state is endoderm, mesoderm or ectoderm can be evaluated.

In the cell differentiation state evaluation method according to the present invention, the stem cells may be pluripotent stem cells such as ES cells (embryonic stem cells) or iPS cells (induced pluripotent stem cells).

The abundances of the indicator substances in the culture supernatant used in the cell differentiation state evaluation method according to the present invention may be obtained using any method. Examples of representative methods include a liquid chromatography analysis method and a mass spectrometry method.

### EFFECT OF INVENTION

According to the cell differentiation state evaluation method according to the present invention, a differentiation state of cells can be noninvasively evaluated without the need of destroying the cells as in a conventional method. As a result, after the evaluation of the differentiation state, it is possible to use the test cells as a cell source or the like for regenerative medicine. Further, even in a case of evaluating temporal change of the differentiation state, it is not necessary to perform complicated work such as culturing multiple culture dishes in parallel as in a conventional method, and it is possible to easily evaluate change with time of the differentiation state with cells in the same culture dish as targets. Further, by performing evaluation based on abundances of two or more kinds of indicator substances in the culture supernatant, the differentiation state of the test cells can be evaluated with higher accuracy. By performing evaluation based on abundances of two or more kinds of indicator substances, in addition to evaluation of whether or not the test cells are in a differentiated state or in an undifferentiated state, it is also possible to evaluate directionality of differentiation of the test cells that have been evaluated as being in a differentiated state.

### BRIEF DESCRIPTION OF DRAWINGS

FIG. 1A A schematic diagram describing a cell differentiation state evaluation method in an example of the present invention (Part 1).

FIG. 1B A schematic diagram describing the cell differentiation state evaluation method in the example of the present invention (Part 2).

FIG. 2A Graphs showing temporal changes of abundances of substances obtained by LC-MS analysis of culture supernatants in the example (Part 1).

FIG. 2B Graphs showing temporal changes of abundances of substances obtained by LC-MS analysis of culture supernatants in the example (Part 2).

FIG. 2C Graphs showing temporal changes of abundances of substances obtained by LC-MS analysis of culture supernatants in the example (Part 3).

FIG. 2D Graphs showing temporal changes of abundances of substances obtained by LC-MS analysis of culture supernatants in the example (Part 4).

FIG. 3A Graphs showing temporal changes of abundances of substances obtained by GC-MS analysis of culture supernatants in the example (Part 1).

FIG. 3B Graphs showing temporal changes of abundances of substances obtained by GC-MS analysis of culture supernatants in the example (Part 2).

FIG. 4A Graphs showing results of principal component analysis of abundances of substances in samples of Day 2-Day 4 of culture in the example (Part 1).

FIG. 4B Graphs showing results of principal component analysis of abundances of substances in samples of Day 2-Day 4 of culture in the example (Part 2).

### MODE FOR CARRYING OUT THE INVENTION

In a cell differentiation state evaluation method according to the present invention, a differentiation state of test cells is evaluated based on an abundance of a biomarker (indicator substance) in a culture supernatant of the test cells.

As the test cells, stem cells, typically pluripotent stem cells such as ES cells or iPS cells can be used. Further, cells differentiation-induced from the stem cells can also be used as test cells. When stem cells for which a differentiation state is unknown are test cells, whether or not the test cells are in a differentiated state or in an undifferentiated state can be determined using the evaluation method of the present invention. When cells differentiation-induced from stem cells are test cells, whether or not cells in an undifferentiated state are mixed can be determined using the evaluation method of the present invention.

As a culture medium used for culturing test cells, a culture medium such as DMEM/F12 that is generally used for culturing stem cells, or a culture medium (such as mTeSR1, and TeSR-E8) containing the DMEM/F12 as a main component can be used. Table 1 shows components of DMEM/F12.

TABLE 1
Components

Amino Acids
Glycine
L-Alanine
L-Arginine hydrochloride
L-Asparagine-H2O
L-Aspartic acid
L-Cysteine hydrochloride-H2O
L-Cystine 2 HCl
L-Glutamic Acid
L-Glutamine
L-Histidine hydrochloride-H2O
L-Isoleucine
L-Leucine
L-Lysine hydrochloride
L-Methionine
L-Phenylalanine
L-Proline
L-Serine
L-Threonine
L-Tryptophan
L-Tyrosine disodium salt dihydrate
L-Valine
Vitamins
Biotin
Choline chloride
D-Calcium pantothenate
Folic Acid
Niacinamide
Pyridoxine hydrochloride
Riboflavin
Thiamine hydrochloride
Vitamin B12
I-Inositol
Inorganic Salts
Calcium Chloride (CaCl2) (anhyd.)
Cupric sulfate (CuSO4—5H2O)
Ferric Nitrate (Fe(NO3)3″9H2O)
Ferric sulfate (FeSO4—7H2O)
Magnesium Chloride (anhydrous)
Magnesium Sulfate (MgSO4) (anhyd.)
Potassium Chloride (KCl)
Sodium Bicarbonate (NaHCO3)
Sodium Chloride (NaCl)
Sodium Phosphate dibasic (Na2HPO4) anhydrous
Sodium Phosphate monobasic (NaH2PO4—H2O)
Zinc sulfate (ZnSO4—7H2O)
Others
D-Glucose (Dextrose)
Hypoxanthine Na
Linoleic Acid
Lipoic Acid
Phenol Red
Putrescine 2HCl
Sodium Pyruvate
Thymidine

Examples of the biomarker include ornithine, 2-aminoadipic acid, deoxycytidine, glutamic acid, tryptophan, asparagine, alanine, cystine, hypoxanthine, uridine aspartic acid, arginine, 2-hydroxybutyric acid, 2-hydroxyisovaleric acid, 3-hydroxyisovaleric acid, urea, 4-hydroxybenzoic acid, 4-aminobenzoic acid, ribonic acid, kynurenine, cystathionine, threonic acid, pyruvic acid, putrescine, ascorbic acid, riboflavin, serine, cysteine, orotic acid, and citric acid.

In a culture supernatant of cells in an undifferentiated state and in a culture supernatant of cells in a differentiated state, abundances of these biomarkers are different. Therefore, by measuring abundances of these biomarkers in a culture supernatant, whether or not the cells are in a differentiated state or in an undifferentiated state can be determined. It is also possible to evaluate a differentiation state by measuring an abundance of a biomarker in a culture supernatant of control cells for which a differentiation state is known, and comparing an abundance of the indicator substance in a culture supernatant of test cells with the abundance of the indicator substance in the culture supernatant of the control cells.

When the differentiation state of the control cells is known, the control cells may be undifferentiated cells or differentiated cells. For differentiated cells, an abundance of a biomarker in a culture supernatant may vary due to a difference in a degree of progression of differentiation, a difference in directionality of differentiation, and the like. Therefore, undifferentiated cells are preferably used as control cells. Comparison of an abundance of a biomarker in a culture supernatant of test cells and an abundance of a biomarker in a culture supernatant of control cells is performed, for example, by obtaining a ratio of the two. When this ratio is greater than or equal to a predetermined threshold (the threshold is greater than 1) or less than or equal to a predetermined threshold (the threshold is less than 1), it can be determined that the cells are in a differentiated state. It is also possible that, when the ratio of the abundance of the biomarker in the culture supernatant of the test cells to the abundance of the biomarker in the culture supernatant of the control cells is the same as the predetermined threshold, it is determined that the cells are in an undifferentiated state, and, when the ratio exceeds the threshold or is less than the threshold, it is determined that the cells are in a differentiated states. When cells differentiation-induced from stem cells are the test cells, the term “undifferentiated state” may include a case where cells in an undifferentiated state are mixed in cells in a differentiated state.

In a preferred embodiment of the cell differentiation state evaluation method according to the present invention, a differentiation state of test cells is evaluated based on abundances of two or more kinds of biomarkers in a culture supernatant of the test cells. By combining two or more kinds of biomarkers, the differentiation state can be evaluated with higher accuracy. Further, by combining two or more kinds of biomarkers, in addition to a differentiation state of cells, whether the cells in a differentiated state are differentiated into endoderm, mesoderm, or ectoderm cells, in other words, directionality of differentiation, can be evaluated.

Two or more kinds of biomarkers can be arbitrarily selected from the above biomarkers. Among the above biomarkers, examples of biomarkers useful for evaluating directionality of differentiation include kynurenine, 2-aminoadipic acid, ornithine, deoxycytidine, tryptophan, cystathionine, hypoxanthine, and uridine. It is preferable to perform evaluation based on abundances of two or more kinds of biomarkers among the above biomarkers.

Examples of method for evaluating a differentiation state based on abundances of two or more kinds of biomarkers include: a method in which change with culture time of an abundance of each biomarker in a culture supernatant of test cells is analyzed; a method in which abundances of two or more kinds of biomarkers are subjected to multivariate analysis; a method in which ratios of abundances of two of more kinds of indicator substances are obtained; and the like. It is also possible to perform evaluation by combining these methods. Determination based on abundances of two kinds of biomarkers may be performed, for example, by plotting an abundance (or index value) of a first biomarker as a horizontal axis and an abundance (or index value) of a second biomarker as a vertical axis. When evaluation is performed based on abundances of three or more kinds of biomarkers, multivariate analysis is useful.

An example of two kinds of biomarkers is a combination of a metabolic product and its precursor in an anteroposterior relation of a metabolic pathway. By performing evaluation based on a ratio of a metabolic product to a precursor, evaluation accuracy of directionality of differentiation is improved. A precursor of a metabolic pathway is not limited to a metabolite with respect to a metabolic product, but may also be a substance of two or more before in the metabolic pathway. Examples of a combination of a precursor and a metabolic product include a combination of tryptophan and kynurenine, a combination of ornithine and putrescine, a combination of arginine and ornithine, and the like. A type of a metabolic pathway is not particularly limited, and combinations of a precursor and a metabolic product are not limited to the above combinations.

In a case where a ratio of a metabolic product to a precursor is obtained, evaluation may be performed based on a ratio of a metabolic product to a precursor in a culture supernatant of test cells, or a ratio of an abundance of a metabolic product to an abundance of a precursor in a culture supernatant of control cells may be compared with a ratio of an abundance of the metabolic product to an abundance of the precursor in a culture supernatant of the test cells. Further, it is also possible to perform evaluation by obtaining a ratio of an abundance of a metabolic product in a culture supernatant of control cells to an abundance of the metabolic product in a culture supernatant of test cells, and a ratio of an abundance of a precursor in a culture supernatant of the control cells to an abundance of the precursor in a culture supernatant of the test cells, and comparing these ratios.

As a method for measuring the abundances of the biomarkers in a culture supernatant, quantitative analysis using mass spectrometry, in particular, quantitative analysis using a liquid chromatography mass spectrometer (LC-MS) or a gas chromatography mass spectrometer (GC-MS) can be suitably used. However, the method for measuring the abundances of the biomarkers in a culture supernatant is not limited to this. For example, it is also possible that a sample obtained by subjecting a culture supernatant to a predetermined pretreatment is flowed together with an eluent into a column of a liquid chromatography (LC) apparatus, and components separated and eluted by the column are detected using a detector such as an ultraviolet-visible spectroscopic detector or an infrared spectroscopic detector, and, from results of the detection, abundances of the biomarkers contained in the sample are obtained. Further, it is also possible that a reagent or the like that specifically causes a biomarker to develop a color or to emit light is added to the culture supernatant, and an abundance of the biomarker is obtained based on an intensity of the color development or the light emission.

### EXAMPLE

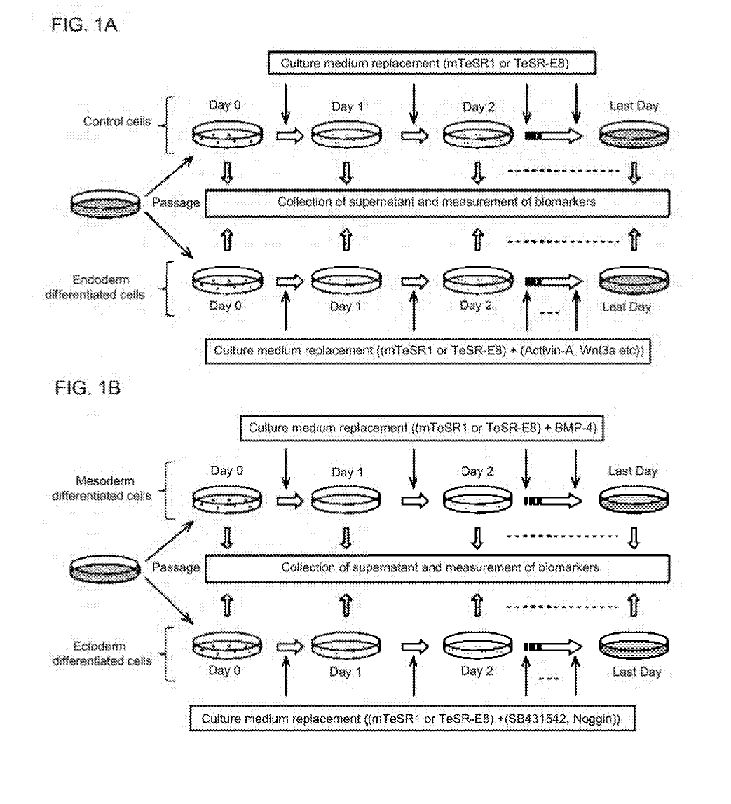

In the following, an example of evaluation of a differentiation state of cells using the method of the present invention is described. FIGS. 1A and 1B are schematic diagrams illustrating procedures of a cell differentiation state evaluation method of the present example.

In the present example, human iPS cell lines PFX #9 were used. Further, cells imparting differentiation induction stimulation to the human iPS cell lines were used as test cells, and cells that do not impart differentiation induction stimulation to the human iPS cell lines (that is, cells that maintain an undifferentiated state) were used as control cells. In the following, procedures from cell culture to analysis of a culture supernatant in the present example are described.

### Culture of Control Cells and Collection of Culture Supernatant

Culture was performed by subculturing the PFX #9 lines in three culture dishes (diameter: 60 mm) coated with Vitronectin-N (Life Technologies Corporation) (in FIG. 1A, for simplicity, only one culture dish is illustrated). As a culture medium, mTeSR1 (modified Tenneille Serum Replacer 1, STEMCELL Technologies Corporation) or TeSR-E8 (Tenneille Serum Replacer E8, STEMCELL Technologies Corporation) was used, and culture medium replacement was performed daily. Components of mTeSR1 are shown in Table 2, and components of TeSR-E8 are shown in Table 3. The day when the subculture (passage) of the cells was performed was taken as Day 0, and the culture was continued for 6 days. A culture supernatant collected from a culture dish when the culture medium was replaced each day was used as a sample for mass spectrometry.

TABLE 2
Components

DMEM/F12
NaHCO3
L-Ascorbic Acid
Selenium
Transferrin
Insulin
FGF2
TGF-β
Bovine Serum Albumin (BSA)
Glutathione
Trace Elements
β-mercaptoethanol
Pipecolic acid
GABA
Lithium Chloride
Defined Lipids

TABLE 3
Components

DMEM/F12
NaHCO3
L-Ascorbic Acid
Selenium
Transferrin
Insulin
FGF2
TGF-β

### Culture of Endoderm Differentiated Cells and Collection of Culture Supernatant

Culture was performed by subcultering the PFX #9 lines in three culture dishes (diameter; 60 mm) coated with Vitronectin-N (in FIG. 1A, for simplicity, only one culture dish is illustrated). As a culture medium, mTeSR1 or TeSR-E8 was used, and culture medium replacement was performed daily, and culture was continued until a confluent state was reached. The day when the passage was performed was taken as Day 0. On Day 1, the culture medium was replaced with a culture medium obtained by adding Activin-A (PeproTech Corporation), Wnt3a (Wingless-type MMTV integration site family, member 3A, PeproTech Corporation), and BMP-4 (Bone Morphogenetic Proteins-4, PeproTech Corporation) such that final concentrations of Activin-A, Wnt3a, and BMP-4 were respectively 100 ng/mL, 40 ng/mL, and 0.5 ng/mL. On Day 2 and later, endoderm differentiation induction stimulation was performed by replacing the culture medium with a culture medium obtained by adding Activin-A and BMP-4 such that final concentrations of Activin-A and BMP-4 were respectively 100 ng/mL and 0.5 ng/mL. The day when the subculture (passage) of the cells was performed was taken as Day 0, and the culture was continued for 6 days. A culture supernatant collected from a culture dish when the culture medium was replaced each day was used as a sample for mass spectrometry.

### Culture of Mesoderm Differentiated Cells and Collection of Culture Supernatant

Culture was performed by subculturing the PFX #9 lines in three culture dishes (diameter: 60mm) coated with Vitronectin-N (in FIG. 1B, for simplicity, only one culture dish is illustrated). As a culture medium, mTeSR1 or TeSR-E8 was used, and culture medium replacement was performed daily, and culture was continued until a confluent state was reached. The day when the passage was performed was taken as Day 0. On Day 1 and later, mesoderm differentiation induction stimulation was performed by replacing the culture medium with a culture medium obtained by adding BMP-4 such that a final concentration of BMP-4 was 40 ng/mL. The day when the subculture (passage) of the cells was performed was taken as Day 0, and the culture was continued for 6 days. A culture supernatant collected from a culture dish when the culture medium was replaced each day was used as a sample for mass spectrometry.

### Culture of Ectoderm Differentiated Cells and Collection of Culture Supernatant

Culture was performed by subculturing the PFX #9 lines in three culture dishes (diameter: 60 mm) coated with Vitronectin-N (in FIG. 1B, for simplicity, only one culture dish is illustrated). As a culture medium, mTeSR1 or TeSR-E8 was used, and culture medium replacement was performed daily, and culture was continued until a confluent state was reached. The day when the passage was performed was taken as Day 0. On Day 1 and later, ectoderm differentiation induction stimulation was performed by replacing the culture medium with a culture medium obtained by adding SB431542 (4-[4-(1,3-benzodioxol-5-yl)-5-(2-pyridinyl)-1H-imidazol-2-yl]benzamide, Wako Pure Chemical Industries, Ltd.) and Noggin (PeproTech Corporation) such that final concentrations of SB431542 and Noggin were respectively 10 μM and 500 ng/ml. The day when the subculture (passage) of the cells was performed was taken as Day 0, and the culture was continued for 6 days. A culture supernatant collected from a culture dish when fee culture medium was replaced each day was used as a sample for mass spectrometry,

### Analysis Using LC-MS

20 μL of 0.5 mM isopropyl malic acid aqueous solution as an internal standard substance was added to 100 μL of the above-described sample, and, after mixing, 200 μL of acetonitrile was added to remove protein. Thereafter, the sample was subjected to centrifugal separation (15,000 rpm, room temperature, 15 minutes), and a supernatant was collected, and was diluted 10-fold with ultrapure water (Milli-Q (registered trademark) water, Merck Corporation) and was then subjected to LC-MS analysis. The LC-MS analysis was performed according to analysis conditions recorded in “LC/MS/MS method package cell culture profiling” (hereinafter abbreviated as “MP”) provided by Shimadzu Corporation. The MP is a collection of analysis condition parameters for analyzing, using LC-MS, compounds contained in the culture medium and metabolites secreted from the cells. Identification of a compound was performed based on that a difference between a retention time of a standard product registered in MP and a retention time of a compound in a sample was within ±0.3 minutes, that peaks of both quantification ions and confirmation ions were detected, and that an intensity value was 1000 or more. Further, quantification of a compound was performed using a method in which a mass chromatogram area for ions (quantification ions) characteristic to each compound in a sample was calculated.

### Analysis Using GC-MS

10 μL of 0.5 mg/mL isopropyl malic acid aqueous solution as an internal standard substance was added to 100 μL of the above-described sample, and, after mixing, 200 μL of acetonitrile was added to remove protein. Thereafter, the sample was subjected to centrifugal separation (15,000 rpm, room temperature, 15 minutes), and 100 μL of a supernatant was collected, and drying under a reduced pressure was performed. By incubating each sample in a pyridine solution containing methoxyamine hydrochloride, methoximation of a compound in the sample was performed. Further, MSTFA (N-methyl-N-trimethylsilyl trifluoroacetamide) was added to each sample to trimethylsilylate (derivative) a compound in the sample. Then, these samples subjected to derivatization treatment were subjected to analysis using GC-MS.

In the GC-MS analysis, “Smart Metabolites Database” (hereinafter abbreviated as “DB”) provided by Shimadzu Corporation was used. The DB is a collection of data obtained by analyzing, using GC-MS, various standard products that have been subjected to the same treatment as the above-described derivatization treatment. Identification of a compound was performed using, as indicators, whether or not a difference between a retention index set in the DB (a relative value of a retention time) and a retention index of a derivatized compound in a sample was within ±5, and whether or not both quantification ions and confirmation ions set in the database were detected for a derivatized compound in the sample. On the other hand, quantification of a compound was performed using a method in which a mass chromatogram area for ions characteristic to each derivatized compound in a sample was calculated according to conditions set in the database.

### Data Analysis

With respect to each of culture supernatants collected from Day 1 to Day 6 of the culture, a value (area ratio) obtained by dividing a quantitative value (area value) of each compound obtained by the LC-MS analysis and the GC-MS analysis by a quantitative value (area value) or the internal standard substance was calculated, and this was taken as an index value of an abundance of the each compound in the culture supernatant. For each of the undifferentiated maintenance cells (control cells), the endoderm differentiated cells, the mesoderm differentiated cells, and the ectoderm differentiated cells, index values of abundances of an identified compound were plotted in a coordinate system in which a horizontal axis represents the number of culture days and a vertical axis represents an index value. Further, a compound for which a change in an index value of an abundance during the course of the culture was clearly different from that of the control cells was selected. Specifically, during a time period from Day 3 to Day 6 of the culture, when an average value of the index values of the control cells was A and an average value of the index values of the test cells was B, a compound for which A/B or B/A was 1.5 or more was selected.

Further, in order to select an effective compound for evaluating a state of cells at an initial stage of the culture, multivariate analysis was performed. Specifically, for the samples during a time period from Day 2 to Day 4 of the culture, the index values calculated above wens input to multivariate analysis software (R version 3.1.2), and principal component analysis was performed. Based on the results of the analysis, a combination of compounds capable of distinguishing between the undifferentiated maintenance cells, the endoderm differentiated cells, the mesoderm differentiated cells, and the ectoderm differentiated cells was selected.

### Results

As a result, compounds for which a change in an index value of an abundance during the course of the culture in a culture supernatant of the control cells is clearly different from that in a culture supernatant of the test cells for which differentiation induction stimulation has been performed are shown in the following Tables 4-24. In these tables, a sample for which a background of a numerical value is shaded indicates a sample for which it is determined that there is a change relative to the index value of the control cells.

TABLE 4
Kynurenine

| | mTeSR1 | | | TeSR-E8 | | |
|---|---|---|---|---|---|---|
| | | Mesoderm | Ectoderm | | Mesoderm | Ectoderm |
| | Endoderm Differentiation | Differentiation | Differentiation | Endoderm Differentiation | Differentiation | Differentiation |
| Day 3 | 0.18893 | 0.21818 | 0.73990 | 0.05575 | 0.11670 | 0.63681 |
| Day 4 | 0.12858 | 0.14785 | 0.59158 | 0.01649 | 0.03062 | 0.33124 |
| Day 5 | 0.19073 | 0.09843 | 0.46329 | 0.01260 | 0.01442 | 0.13219 |
| Day 6 | 0.35260 | 0.03593 | 0.38332 | 0.01525 | 0.00786 | 0.06775 |

From Table 4, it is found that, for kynurenine, due to differentiation induction, an abundance in a culture supernatant of the test cells is decreased relative to an abundance in a culture supernatant of the control cells. Therefore, in a case where undifferentiated cells are used as the control cells, when a ratio ((test cells)/(control cells)) of an abundance in a culture supernatant of the test cells to an abundance of kynurenine in a culture supernatant of the control cells is equal to or less than a predetermined threshold, it can be determined that the test cells are in a differentiated state.

TABLE 5
Ornithine

| | mTeSR1 | | | TeSR-E8 | | |
|---|---|---|---|---|---|---|
| | | Mesoderm | Ectoderm | | Mesoderm | Ectoderm |
| | Endoderm Differentiation | Differentiation | Differentiation | Endoderm Differentiation | Differentiation | Differentiation |
| Day 3 | 0.85153 | 0.61255 | 0.92215 | 0.47613 | 0.40499 | 1.04040 |
| Day 4 | 0.82127 | 0.58431 | 0.96502 | 0.35876 | 0.16613 | 0.93894 |
| Day 5 | 0.68689 | 0.59170 | 0.88162 | 0.35903 | 0.12975 | 0.90758 |
| Day 6 | 0.67821 | 0.49754 | 0.77119 | 0.30423 | 0.14435 | 0.91217 |

From Table 5, it is found that, for ornithine, due to differentiation induction, an abundance in a supernatant of the test cells is decreased as compared to the control cells. Therefore, in the case where undifferentiated cells are used as the control cells, when a ratio ((test cells)/(control cells)) of an abundance in a culture supernatant of the test cells to an abundance of ornithine in a culture supernatant of the control cells is equal to or less than a predetermined threshold, it can be determined that the test cells are in a differentiated state.

TABLE 6
2-Aminoadipic acid

| | mTeSR1 | | | TeSR-E8 | | |
|---|---|---|---|---|---|---|
| | | Mesoderm | Ectoderm | | Mesoderm | Ectoderm |
| | Endoderm Differentiation | Differentiation | Differentiation | Endoderm Differentiation | Differentiation | Differentiation |
| Day 3 | 0.05193 | 0.58617 | 1.80826 | 0.26223 | 0.34349 | 7.45760 |
| Day 4 | 0.02984 | 0.47606 | 2.11081 | 0.20057 | 0.20629 | 7.26711 |
| Day 5 | 0.03172 | 0.50292 | 2.40063 | 0.18995 | 0.22813 | 6.35465 |
| Day 6 | 0.07205 | 0.70721 | 3.13504 | 0.18617 | 0.10794 | 3.68514 |

From Table 6, it is found that, for 2-aminoadipic acid, for the test cells differentiation-induced into endoderm and mesoderm, an abundance in a supernatant is decreased as compared to the control cells. On the other hand, it is found that, for the test cells differentiation-induced into ectoderm, an abundance in a supernatant is increased as compared to the control cells for all Day 3-Day 6 of the culture. Therefore, in the case where undifferentiated cells are used as the control cells, when a ratio ((test cells)/(control cells)) of an abundance in a culture supernatant of the test cells to an abundance of 2-aminoadipic acid in a culture supernatant of the control cells is equal to or less than a predetermined threshold, it can be determined that the test cells are differentiated toward endoderm or mesoderm, and when the ratio is equal to or greater than the threshold, it can be determined that the test cells are differentiated toward ectoderm.

TABLE 7
Deoxycytidine

| | mTeSR1 | | | TeSR-E8 | | |
|---|---|---|---|---|---|---|
| | | Mesoderm | Ectoderm | | Mesoderm | Ectoderm |
| | Endoderm Differentiation | Differentiation | Differentiation | Endoderm Differentiation | Differentiation | Differentiation |
| Day 3 | 0.12568 | 1.67146 | 0.97323 | 0.27835 | 0.95542 | 0.38476 |
| Day 4 | 0.05935 | 2.03705 | 1.32781 | 0.11147 | 1.62561 | 0.91033 |
| Day 5 | 0.07383 | 3.15055 | 1.35494 | 0.25834 | 4.85835 | 3.23319 |
| Day 6 | 0.11886 | 3.18575 | 0.84337 | 0.24061 | 4.60101 | 2.07900 |

From Table 7, it is found that, for deoxycytidine, for the test cells differentiation-induced into endoderm, an abundance in a supernatant is decreased as compared to the control cells. On the other hand, it is found that, for the test cells differentiation-induced into mesoderm and some of the test cells differentiation-induced into ectoderm, an abundance in a supernatant is increased as compared to the control cells. Therefore, in the case where undifferentiated cells are used as the control cells, when a ratio ((test cells)/(control cells)) of an abundance in a culture supernatant of the test cells to an abundance of deoxycytidine in a culture supernatant of the control cells is equal to or less than a predetermined threshold, it can be determined that the test cells are differentiated toward endoderm, and when the ratio is equal to or greater than the threshold, it can be determined that the test cells are differentiated toward mesoderm or ectoderm.

TABLE 8
Glutamic acid

| | mTeSR1 | | | TeSR-E8 | | |
|---|---|---|---|---|---|---|
| | | Mesoderm | Ectoderm | | Mesoderm | Ectoderm |
| | Endoderm Differentiation | Differentiation | Differentiation | Endoderm Differentiation | Differentiation | Differentiation |
| Day 3 | 1.04830 | 1.03500 | 0.91965 | 1.17905 | 1.03364 | 0.98334 |
| Day 4 | 1.25414 | 1.02633 | 0.98379 | 1.66323 | 0.95813 | 1.04074 |
| Day 5 | 1.64594 | 0.92395 | 1.24662 | 1.91679 | 0.75864 | 1.03077 |
| Day 6 | 1.46709 | 0.80920 | 0.87426 | 1.53753 | 1.07436 | 1.02387 |

From Table 8, it is found that, for glutamic acid, an abundance in a supernatant of the test cells differentiation-induced into endoderm is increased as compared to the control cells. Therefore, in the case where undifferentiated cells are used as the control cells, when a ratio ((test cells)/(control cells)) of an abundance in a culture supernatant of the test cells to an abundance of glutamic acid in a culture supernatant of the control cells is equal to or greater than a predetermined threshold, it can be determined that the test cells are differentiated toward endoderm.

TABLE 9
Tryptophan

| | mTeSR1 | | | TeSR-E8 | | |
|---|---|---|---|---|---|---|
| | | Mesoderm | Ectoderm | | Mesoderm | Ectoderm |
| | Endoderm Differentiation | Differentiation | Differentiation | Endoderm Differentiation | Differentiation | Differentiation |
| Day 3 | 1.09811 | 1.21260 | 1.10550 | 1.02888 | 1.03523 | 1.03966 |
| Day 4 | 2.70456 | 3.29632 | 2.55021 | 1.26312 | 1.34472 | 1.30520 |
| Day 5 | 61.95333 | 85.32587 | 49.71053 | 2.44475 | 2.25226 | 2.26845 |
| Day 6 | 50.22615 | 105.71030 | 39.13180 | 3.10929 | 3.15314 | 3.11552 |

From Table 9, it is found that, for tryptophan, due to differentiation induction, an abundance in a supernatant of the test cells is increased as compared to an abundance in a supernatant of the control cells. Therefore, in the case where undifferentiated cells are used as the control cells, when a ratio ((test cells)/(control cells)) of an abundance in a culture supernatant of the test cells to an abundance of tryptophan in a culture supernatant of the control cells is equal to or greater than a predetermined threshold, it can be determined that the test cells are in a differentiated state.

TABLE 10
Cystathionine

| | mTeSR1 | | | TeSR-E8 | | |
|---|---|---|---|---|---|---|
| | | Mesoderm | Ectoderm | | Mesoderm | Ectoderm |
| | Endoderm Differentiation | Differentiation | Differentiation | Endoderm Differentiation | Differentiation | Differentiation |
| Day 3 | 1.16223 | 0.82639 | 0.76152 | 0.91514 | 0.91300 | 0.92984 |
| Day 4 | 1.02042 | 0.53625 | 0.76685 | 0.67659 | 0.48016 | 0.75582 |
| Day 5 | 0.77199 | 0.21684 | 0.74793 | 0.46001 | 0.38621 | 0.68651 |
| Day 6 | 0.78068 | 0.22021 | 0.77175 | 0.39274 | 0.39633 | 0.84683 |

From Table 10, it is found that, for cystathionine, due to differentiation induction, an abundance in a supernatant of the test cells is generally decreased as compared to an abundance in a supernatant of the control cells. Therefore, in the case where undifferentiated cells are used as the control cells, when a ratio ((test cells)/(control cells)) of an abundance in a culture supernatant of the test cells to an abundance of cystathionine in a culture supernatant of the control cells is equal to or less than a predetermined threshold, it can be determined that the test cells are in a differentiated state.

TABLE 11
Alanine

| | mTeSR1 | | | TeSR-E8 | | |
|---|---|---|---|---|---|---|
| | | Mesoderm | Ectoderm | | Mesoderm | Ectoderm |
| | Endoderm Differentiation | Differentiation | Differentiation | Endoderm Differentiation | Differentiation | Differentiation |
| Day 3 | 0.93195 | 0.80433 | 0.94320 | 0.96237 | 0.77472 | 1.05763 |
| Day 4 | 1.01651 | 0.83640 | 0.92764 | 0.96875 | 0.71357 | 0.97390 |
| Day 5 | 0.85849 | 0.78237 | 0.84835 | 0.78790 | 0.62014 | 0.73593 |
| Day 6 | 0.73327 | 0.61276 | 0.75377 | 0.59205 | 0.56090 | 0.72836 |

From Table 11, it is found that, for alanine, due to differentiation induction, an abundance in a supernatant of the test cells is generally decreased as compared to an abundance in a supernatant of the control cells. Therefore, in the case where undifferentiated cells are used as the control cells, when a ratio ((test cells)/(control cells)) of an abundance in a culture supernatant of the test cells to an abundance of alanine in a culture supernatant of the control cells is equal to or less than a predetermined threshold, it can be determined that the test cells are in a differentiated state.

TABLE 12
Cystine

| | mTeSR1 | | | TeSR-E8 | | |
|---|---|---|---|---|---|---|
| | | Mesoderm | Ectoderm | | Mesoderm | Ectoderm |
| | Endoderm Differentiation | Differentiation | Differentiation | Endoderm Differentiation | Differentiation | Differentiation |
| Day 3 | 0.91086 | 1.06517 | 1.09895 | 0.87903 | 0.92513 | 0.91374 |
| Day 4 | 1.14269 | 1.94349 | 1.11833 | 0.69469 | 1.13958 | 0.92408 |
| Day 5 | 0.79866 | 2.28473 | 0.82651 | 0.47528 | 1.48378 | 0.86547 |
| Day 6 | 0.89009 | 3.74203 | 1.01225 | 0.47205 | 1.37228 | 0.61081 |

From Table 12, it is found that, for cystine, for the test cells differentiation-induced into endoderm and ectoderm, an abundance in a supernatant is generally decreased as compared to the control cells. On the other hand, it is found that, for the test cells differentiation-induced into mesoderm, an abundance in a supernatant is generally increased as compared to the control cells. Therefore, in the case where undifferentiated cells are used as the control cells, when a ratio ((test cells)/(control cells)) of an abundance in a culture supernatant of the test cells to an abundance of cystine in a culture supernatant of the control cells is equal to or less than a predetermined threshold, it can be determined that the test cells are differentiated toward endoderm or ectoderm, and when the ratio is equal to or greater than the threshold, it can be determined that the test cells are differentiated toward mesoderm.

TABLE 13
Hypoxanthine

| | mTeSR1 | | | TeSR-E8 | | |
|---|---|---|---|---|---|---|
| | | Mesoderm | Ectoderm | | Mesoderm | Ectoderm |
| | Endoderm Differentiation | Differentiation | Differentiation | Endoderm Differentiation | Differentiation | Differentiation |
| Day 3 | 0.48051 | 2.98594 | 6.57292 | 0.47858 | 1.47942 | 4.55676 |
| Day 4 | 0.90741 | 3.46975 | 4.66758 | 0.98274 | 5.88716 | 50.41902 |
| Day 5 | 3.76607 | 10.33399 | 0.95857 | 3.32066 | 3.95113 | 40.10208 |
| Day 6 | 2.02114 | 10.69372 | 1.78502 | 0.42838 | 0.58486 | 3.44223 |

From Table 13, it is found that, for hypoxanthine, for the test cells differentiation-induced into mesoderm and ectoderm, an abundance in a supernatant is generally increased as compared to the control cells until Day 5 of the culture. Therefore, for hypoxanthine, in the case where undifferentiated cells are used as the control cells, during a time period from the start to Day 5 of the culture, when a ratio ((test cells)/(control cells)) of an abundance in a culture supernatant of the test cells to an abundance of hypoxanthine in a culture supernatant of the control cells is equal to or greater than a predetermined threshold, it can be determined that the test cells are differentiated toward mesoderm or ectoderm.

TABLE 14
Uridine

| | mTeSR1 | | | TeSR-E8 | | |
|---|---|---|---|---|---|---|
| | | Mesoderm | Ectoderm | | Mesoderm | Ectoderm |
| | Endoderm Differentiation | Differentiation | Differentiation | Endoderm Differentiation | Differentiation | Differentiation |
| Day 3 | 1.27119 | 1.06620 | 1.16740 | 0.95583 | 1.05244 | 0.92484 |
| Day 4 | 1.92446 | 1.72021 | 1.14334 | 2.73917 | 2.51786 | 0.97607 |
| Day 5 | 4.60898 | 3.82077 | 0.83752 | 5.06128 | 1.83698 | 1.53353 |
| Day 6 | 1.33203 | 1.72377 | 0.86755 | 3.26169 | 1.17087 | 1.23247 |

From Table 14, it is found that, for uridine, due to differentiation induction, an abundance in a supernatant of the test cells is increased as compared to an abundance in a supernatant of the control cells. Therefore, in the case where undifferentiated cells are used as the control cells, when a ratio ((test cells)/(control cells)) of an abundance in a culture supernatant of the test cells to an abundance of uridine in a culture supernatant of the control cells is equal to or greater than a predetermined threshold, it can be determined that the test cells are in a differentiated state.

TABLE 15
Aspartic acid

| | mTeSR1 | | | TeSR-E8 | | |
|---|---|---|---|---|---|---|
| | Endoderm | Mesoderm | Ectoderm | Endoderm | Mesoderm | Ectoderm |
| | Differentiation | Differentiation | Differentiation | Differentiation | Differentiation | Differentiation |
| Day 3 | 0.95932 | 0.96292 | 0.94153 | 1.03864 | 1.00146 | 1.01118 |
| Day 4 | 0.99362 | 0.94460 | 0.90931 | 1.16674 | 0.84659 | 0.92238 |
| Day 5 | 1.07273 | 1.01921 | 1.02430 | 1.21030 | 0.63493 | 0.80039 |
| Day 6 | 0.87298 | 0.92329 | 0.66322 | 0.98378 | 0.59203 | 0.75007 |

From Table 15, it is found that, for aspartic acid, due to differentiation induction, an abundance in a supernatant of the test cells is decreased as compared to an abundance in a supernatant of the control cells. Therefore, in the case where undifferentiated cells are used as the control cells, when a ratio ((test cells)/(control cells)) of an abundance in a culture supernatant of the test cells to an abundance of aspartic acid in a culture supernatant of the control cells is equal to or less than a predetermined threshold, it can be determined that the test cells are in a differentiated state.

TABLE 16
Arginine

| | mTeSR1 | | | TeSR-E8 | | |
|---|---|---|---|---|---|---|
| | Endoderm | Mesoderm | Ectoderm | Endoderm | Mesoderm | Ectoderm |
| | Differentiation | Differentiation | Differentiation | Differentiation | Differentiation | Differentiation |
| Day 3 | 0.94176 | 1.04160 | 0.87727 | 1.04290 | 1.05220 | 0.93153 |
| Day 4 | 1.18873 | 1.37727 | 0.97998 | 1.16855 | 1.20608 | 0.96800 |
| Day 5 | 1.72819 | 2.89957 | 1.27474 | 1.42427 | 1.42390 | 1.07959 |
| Day 6 | 2.21368 | 2.85952 | 1.56629 | 1.41294 | 1.51619 | 1.03255 |

From Table 16, it is found that, for arginine, due to differentiation induction, an abundance in a supernatant of the test cells is generally increased as compared to an abundance in a supernatant of the control cells. Therefore, in the case where undifferentiated cells are used as the control cells, when a ratio ((test cells)/(control cells)) of an abundance in a culture supernatant of the test cells to an abundance of arginine in a culture supernatant of the control cells is equal to or greater than a predetermined threshold, it can be determined that the test cells are in a differentiated state.

TABLE 17
2-Hydroxybutyric acid

| | mTeSR1 | | | TeSR-E8 | | |
|---|---|---|---|---|---|---|
| | Endoderm | Mesoderm | Ectoderm | Endoderm | Mesoderm | Ectoderm |
| | Differentiation | Differentiation | Differentiation | Differentiation | Differentiation | Differentiation |
| Day 3 | 1.07430 | 0.95423 | 0.78712 | 0.97805 | 0.96107 | 1.06073 |
| Day 4 | 0.92993 | 0.84393 | 0.62321 | 0.96395 | 1.01651 | 0.89628 |
| Day 5 | 0.48145 | 0.86498 | 0.69611 | 0.75546 | 0.73369 | 0.73792 |
| Day 6 | 0.47994 | 0.46706 | 0.44935 | 0.54717 | 0.43526 | 0.59593 |

From Table 17, it is found that, for 2-hydroxybutyric acid, due to differentiation induction, an abundance in a supernatant of the test cells is decreased as compared to an abundance in a supernatant of the control cells. Therefore, in the case where undifferentiated cells are used as the control cells, when a ratio ((test cells)/(control cells)) of an abundance in a culture supernatant of the test cells to an abundance of 2-hydroxybutyric acid in a culture supernatant of the control cells is equal to or less than a predetermined threshold it can be determined that the test cells are in a differentiated state.

TABLE 18
2-Hydroxyisovaleric acid

| | mTeSR1 | | | TeSR-E8 | | |
|---|---|---|---|---|---|---|
| | Endoderm | Mesoderm | Ectoderm | Endoderm | Mesoderm | Ectoderm |
| | Differentiation | Differentiation | Differentiation | Differentiation | Differentiation | Differentiation |
| Day 3 | 1.27255 | 0.98944 | 1.10055 | 1.30692 | 1.38960 | 1.42050 |
| Day 4 | 1.79894 | 0.75779 | 1.24476 | 1.35047 | 0.83504 | 1.01369 |
| Day 5 | 0.61928 | 0.29113 | 0.41913 | 0.92482 | 0.53223 | 0.64369 |
| Day 6 | 0.92244 | 0.46552 | 0.69112 | 0.71194 | 0.62099 | 0.73549 |

From Table 18, it is found that, for 2-hydroxyisovaleric acid, due to differentiation induction, an abundance in a supernatant of the test cells is generally decreased as compared to the control cells. Therefore, in the case where undifferentiated cells are used as the control cells, when a ratio ((test cells)/(control cells)) of an abundance in a culture supernatant of the test cells to an abundance of 2-hydroxyisovaleric acid in a culture supernatant of the control cells is equal to or less than a predetermined threshold, it can be determined that the test cells are in a differentiated state.

TABLE 19
3-Hydroxyisovaleric acid

| | mTeSR1 | | | TeSR-E8 | | |
|---|---|---|---|---|---|---|
| | Endoderm | Mesoderm | Ectoderm | Endoderm | Mesoderm | Ectoderm |
| | Differentiation | Differentiation | Differentiation | Differentiation | Differentiation | Differentiation |
| Day 3 | 1.02519 | 0.85818 | 1.15222 | 1.02921 | 1.18842 | 0.88045 |
| Day 4 | 0.65141 | 1.03118 | 1.13604 | 0.37724 | 0.72505 | 0.37461 |
| Day 5 | 0.71332 | 1.13614 | 1.43775 | 0.27049 | 0.27218 | 0.30534 |
| Day 6 | 1.03230 | 0.60868 | 1.20830 | 0.38348 | 0.17757 | 0.36216 |

From Table 19, it is found that, for 3-hydroxyisovaleric acid, due to differentiation induction, except for some of the test cells differentiation-induced into endoderm and ectoderm, an abundance in a supernatant of the test cells is decreased as compared to an abundance in a supernatant of the control cells. Therefore, in the case where undifferentiated cells are used as the control cells, when a ratio ((test cells)/(control cells)) of an abundance in a culture supernatant of the test cells to an abundance of 3-hydroxyisovaleric acid in a culture supernatant of the control cells is equal to or less than a predetermined threshold, it can be determined that the test cells are in a differentiated state.

TABLE 20
Urea

| | mTeSR1 | | | TeSR-E8 | | |
|---|---|---|---|---|---|---|
| | Endoderm | Mesoderm | Ectoderm | Endoderm | Mesoderm | Ectoderm |
| | Differentiation | Differentiation | Differentiation | Differentiation | Differentiation | Differentiation |
| Day 3 | 0.92310 | 0.70312 | 1.01352 | 0.64805 | 0.66719 | 1.27356 |
| Day 4 | 0.80755 | 0.71764 | 1.07742 | 0.41185 | 0.21851 | 1.00317 |
| Day 5 | 0.65412 | 0.61466 | 0.96093 | 0.38509 | 0.20195 | 0.92338 |
| Day 6 | 0.63476 | 0.50105 | 0.79971 | 0.30602 | 0.12709 | 0.85022 |

From Table 20, it is found that, for urea, due to differentiation induction, except for some of die test cells differentiation-induced into ectoderm, an abundance in a supernatant of the test cells is decreased as compared to an abundance in a supernatant of the control cells. In the case where undifferentiated cells are used as the control cells, when a ratio ((test cells)/(control cells)) of an abundance in a culture supernatant of the test cells to an abundance of urea in a culture supernatant of the control cells is equal to or less than a predetermined threshold, it can be determined that the test cells are in a differentiated state.

TABLE 21
4-Hydroxybenzoic acid

| | mTeSR1 | | | TeSR-E8 | | |
|---|---|---|---|---|---|---|
| | Endoderm | Mesoderm | Ectoderm | Endoderm | Mesoderm | Ectoderm |
| | Differentiation | Differentiation | Differentiation | Differentiation | Differentiation | Differentiation |
| Day 3 | 1.27722 | 0.67413 | 0.88865 | 1.64980 | 0.76347 | 1.10956 |
| Day 4 | 1.11066 | 0.53828 | 0.71423 | 0.67843 | 0.44433 | 0.73755 |
| Day 5 | 0.63066 | 0.51684 | 0.53872 | 0.33834 | 0.37585 | 0.39470 |
| Day 6 | 0.72499 | 0.41491 | 0.67327 | 0.32236 | 0.30864 | 0.31271 |

From Table 21, it is found that, for 4-hydroxybenzoic acid, due to differentiation induction, except for some of the test cells differentiation-induced into endoderm and some of the test cells differentiation-induced into ectoderm, an abundance in a supernatant of the test cells is decreased as compared to an abundance in a supernatant of the control cells. Therefore, in the case where differentiated cells are used as the control cells, when a ratio ((test cells)/(control cells)) of an abundance in a culture supernatant of the test cells to an abundance of 4-hydroxybenzoic acid in a culture supernatant of the control cells is equal to or less than a predetermined threshold, it can be determined that the test cells are in a differentiated state.

TABLE 22
4-Aminobenzoic acid

| | mTeSR1 | | | TeSR-E8 | | |
|---|---|---|---|---|---|---|
| | Endoderm | Mesoderm | Ectoderm | Endoderm | Mesoderm | Ectoderm |
| | Differentiation | Differentiation | Differentiation | Differentiation | Differentiation | Differentiation |
| Day 3 | 1.24586 | 1.49127 | 3.01331 | 1.29965 | 1.04855 | 2.81920 |
| Day 4 | 1.29134 | 1.31592 | 3.01235 | 1.15175 | 1.11488 | 2.41449 |
| Day 5 | 2.61237 | 1.08518 | 1.17458 | 1.16881 | 1.76581 | 3.06917 |
| Day 6 | 1.13642 | 1.05606 | 2.41449 | 1.10461 | 1.09812 | 2.07817 |

From Table 22, it is found that, for 4-aminobenzoic acid, due to differentiation induction, an abundance in a supernatant of the test cells is increased as compared to an abundance in a supernatant of the control cells. Therefore, in the case where undifferentiated cells are used as the control cells, when a ratio ((test cells)/(control cells)) of an abundance in a culture supernatant of the test cells to an abundance of 4-aminobenzoic acid in a culture supernatant of the control cells is equal to or greater than a predetermined threshold, it can be determined that the test cells are in a differentiated state.

TABLE 23
Ribonic acid

| | mTeSR1 | | | TeSR-E8 | | |
|---|---|---|---|---|---|---|
| | Endoderm | Mesoderm | Ectoderm | Endoderm | Mesoderm | Ectoderm |
| | Differentiation | Differentiation | Differentiation | Differentiation | Differentiation | Differentiation |
| Day 3 | 1.06590 | 0.84094 | 1.03030 | 1.08877 | 0.73380 | 1.37594 |
| Day 4 | 1.05338 | 0.68870 | 0.97971 | 1.15439 | 0.48453 | 1.11639 |
| Day 5 | 1.04836 | 0.46572 | 1.03581 | 1.14568 | 0.31384 | 1.14763 |
| Day 6 | 0.96847 | 0.34366 | 0.90276 | 1.11614 | 0.35749 | 1.11192 |

From Table 23, it is found that, for ribonic acid, for the test cells differentiation-induced into mesoderm, an abundance in a supernatant is decreased as compared to the control cells. Therefore, in the case where undifferentiated cells are used as the control cells, when a ratio ((test cells)/(control cells)) of an abundance in a culture supernatant of the test cells to an abundance of ribonic acid in a culture supernatant of the control cells is equal to or less than a predetermined threshold, it can be determined that the test cells are differentiated toward mesoderm.

TABLE 24
Citric acid

| | mTeSR1 | | | TeSR-E8 | | |
|---|---|---|---|---|---|---|
| | Endoderm | Mesoderm | Ectoderm | Endoderm | Mesoderm | Ectoderm |
| | Differentiation | Differentiation | Differentiation | Differentiation | Differentiation | Differentiation |
| Day 3 | 1.06624 | 0.98091 | 0.92673 | 7.21362 | 1.38785 | 1.23408 |
| Day 4 | 1.10012 | 1.59673 | 0.85179 | 5.09693 | 2.15716 | 0.84740 |
| Day 5 | 0.91417 | 2.12469 | 0.93419 | 3.38313 | 3.13229 | 0.58426 |
| Day 6 | 1.00922 | 2.18626 | 1.22431 | 2.23508 | 4.18140 | 0.49242 |

From Table 24, is found that, for citric acid, for the test cells differentiation-induced into mesoderm and some of the test cells differentiation-induced into endoderm, due to differentiation induction, an abundance in a supernatant of the test cells is increased as compared to the control cells. Therefore, in the case where undifferentiated cells are used as the control cells, when a ratio ((test cells)/(control ceils)) of an abundance in a culture supernatant of the test cells to an abundance of citric acid in a culture supernatant of the control cells is equal to or less than a predetermined threshold, it can be determined that the test cells are differentiated toward endoderm or mesoderm.

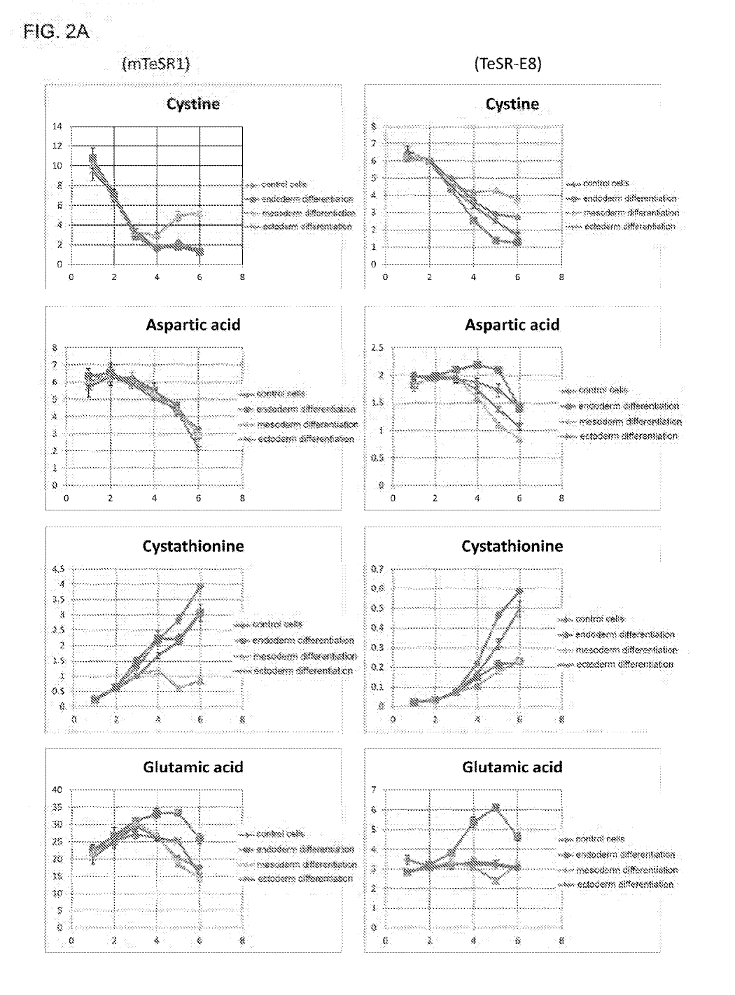

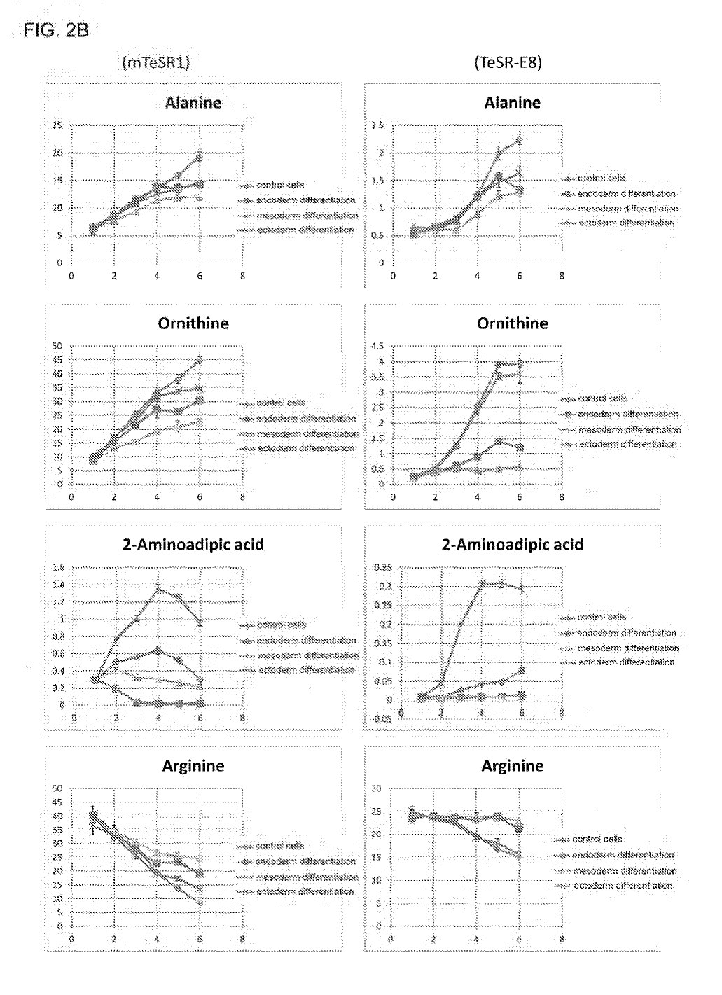

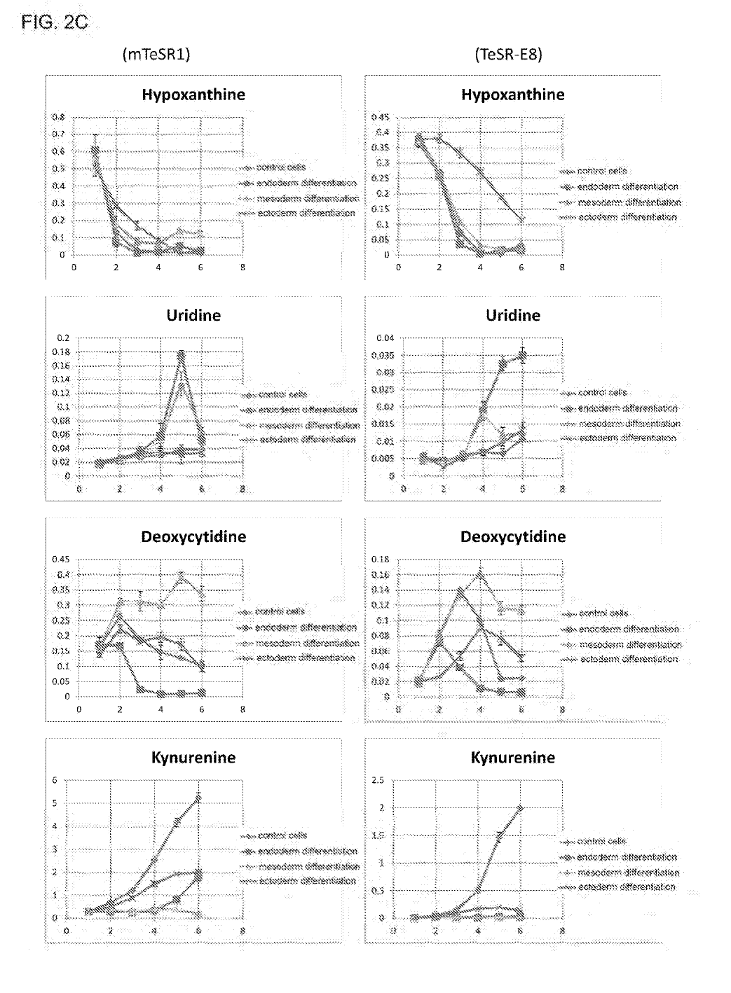

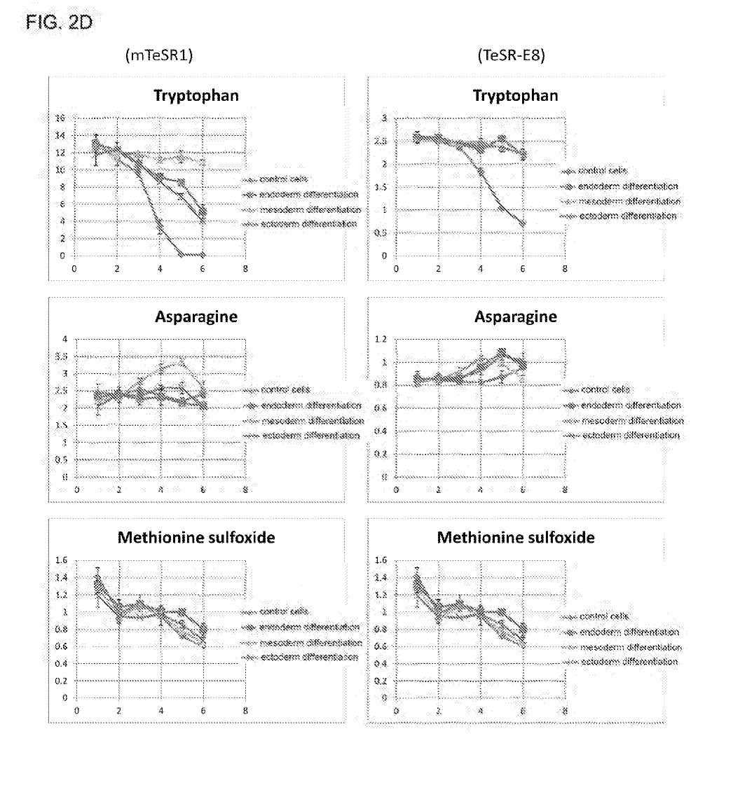

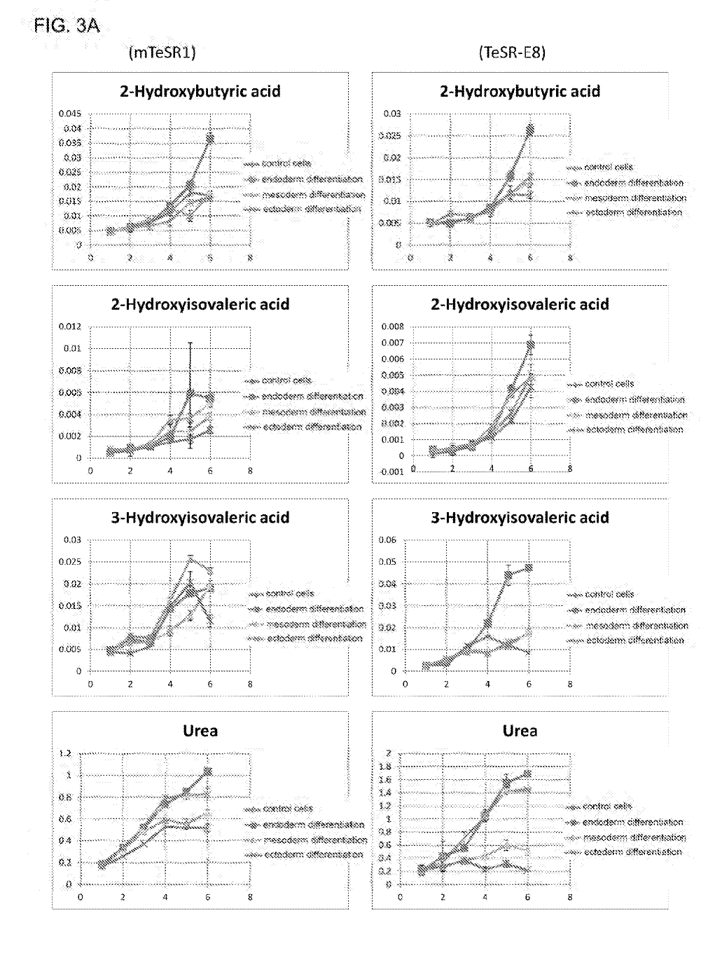

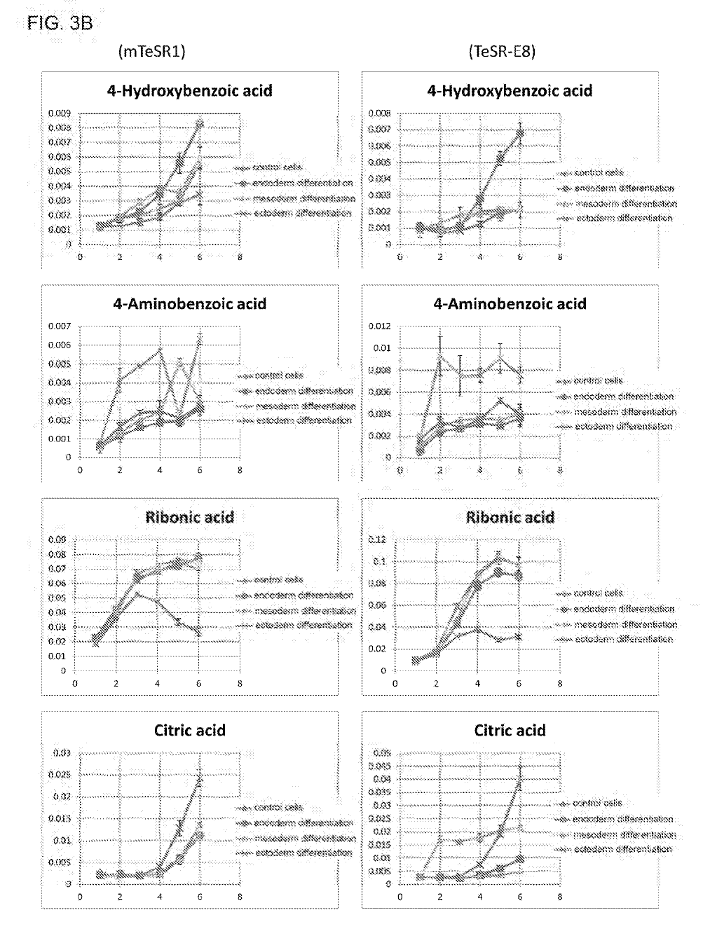

FIGS. 2A, 2B, 2C, 2D, 3A and 3B show changes in abundance of the biomarkers in culture supernatants of Day 1-Day 6 of culture of mTeSR1 lines and TeSR-E8 lines. FIGS. 2A-2D show results of LC-MS analyses, and FIGS. 3A and 3B show results of GC-MS analyses. As is clear from these figures, it is confirmed that, although there are no differences in the abundances of the biomarkers between the test cells and the control cells, for many of the biomarkers, the differences in the abundances between the test cells and the control cells increase as time passes. It is confirmed that the time when the difference in abundance between the control cells and the test cells becomes the largest depends on the kind of the cultured cells and the kind of the biomarker. Further, it is also confirmed that there are differences in abundances of biomarkers depending on the differentiation state of the test cells (the endoderm differentiated cells, the mesoderm differentiated cells, and the ectoderm differentiated cells).

For example, for kynurenine, relative to the test cells, an abundance in a culture supernatant of the control cells greatly increases as culture time passes. For 2-aminoadipic acid, although there are nearly no changes in abundances in culture supernatants of the endoderm differentiated cells and the mesoderm differentiated cells, there are changes (increases) in abundances in culture supernatants of the control cells and the ectoderm differentiated cells, and in particular, there is a large change for the ectoderm differentiated cells. For deoxycytidine, after seeding, there are changes (increases) in abundances in culture supernatants of all the cells. However, for the cells subjected to differentiation induction stimulation to endoderm, an abundance in a culture supernatant greatly decreases as culture time passes. Therefore, by utilizing such a feature of each biomarker regarding a change in an abundance in a culture supernatant, it is possible to determine whether or not the test cells are in a differentiated state or in an undifferentiated state and to evaluate the differentiation directionality (endoderm, mesoderm, or ectoderm) of the test cells that have been determined as being in a differentiated state. In evaluation based on only one kind of a biomarker, it is difficult to evaluate all of endoderm differentiation, mesoderm differentiation and ectoderm differentiation. However, when determination is performed based on abundances of two or more kinds of biomarkers, the differentiation directionality can be more accurately evaluated.

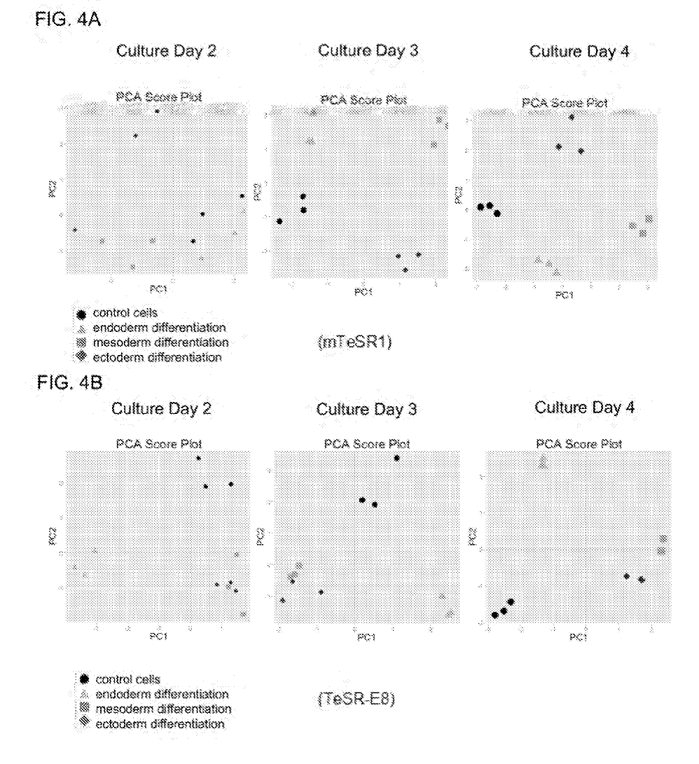

FIG. 4A shows results of principal component analyses of abundances of kynurenine, 2-aminoadipic acid, ornithine, deoxycytidine, tryptophan, cystathionine, hypoxanthine, and uridine in culture supernatants of Day 2-Day 4 of culture of mTeSR1 lines. From this figure, it is found that, on Day 3 of the culture, the undifferentiated state (the control cells) can be clearly distinguished from each of the endoderm differentiated cells, the mesoderm differentiated cells and the ectoderm differentiated cells.

Further, FIG. 4B shows results of principal component analysis of abundances of the biomarkers in culture supernatants of Day 2-Day 4 of culture of TeSR-E8 lines. From this figure, it is found that, on Day 4 of the culture, the undifferentiated state (the control cells) can be clearly distinguished from each of the endoderm differentiated cells, the mesoderm differentiated cells and the ectoderm differentiated cells.

That is, as shown in FIGS. 4A and 4B, it is found that, by using abundances of two or more kinds of biomarkers in a culture supernatant as indicators, it is possible to determine whether or not the test cells are in a differentiated state or in an undifferentiated state, and to evaluate differentiation directionality (endoderm, mesoderm, or ectoderm) of the test cells.

### Comparison With Other Test Methods

Analysis based on cell destructive inspection was performed in order to confirm consistency between analysis of an abundance of a biomarker in a culture supernatant and analysis based on other test methods.

Similar to “the culture of the target cells,” “the culture of the endoderm differentiated cells,” “the culture of the mesoderm differentiated cells” and “the culture of the ectoderm differentiated cells” described above, mTeSR1 or TeSR-E8 was used as a culture medium, culture of cells and differentiation induction stimulation were performed, and the day when passage was performed was taken as Day 0, and cells were collected after Day 4 and after Day 7.

mRNA of the collected cells was extracted using TaqMan HPSC Scorecard Panel (Applied Biosystems Corporation), and 96 kinds of gene expression levels were evaluated using quantitative PCR, and, based on the results, scores of degrees of differentiation into the germ layers were calculated. Analysts scores of the mTeSR1 lines are shown in Table 25, and analysis scores of the TeSR-E8 lines are shown in Table 26. In these tables, for a sample for which a background of a numerical value is shaded, a analysis score is 0.5 or more, and it indicates that the cells are differentiated in a specific direction.

TABLE 25

| | Analysis Score | | |
|---|---|---|---|
| | Endoderm | | Ectoderm |
| | Differen- | Mesoderm | Differen- |
| mTeSR1 | tiation | Differentiation | tiation |
| Control Cells Day 4 | −1.70 | −1.06 | −1.43 |
| Control Cells Day 7 | −2.07 | −1.44 | −1.39 |
| Endoderm Differentiation Day 4 | 0.60 | −0.54 | −1.30 |
| Endoderm Differentiation Day 7 | 0.75 | −0.51 | −0.86 |
| Mesoderm Differentiation Day 4 | −1.57 | 0.53 | −1.41 |
| Mesoderm Differentiation Day 7 | −1.71 | 3.03 | −0.41 |
| Ectoderm Differentiation Day 4 | −1.68 | −0.93 | −0.29 |
| Ectoderm Differentiation Day 7 | −1.24 | −0.63 | 1.60 |

TABLE 26

| | Analysis Score | | |
|---|---|---|---|
| | Endoderm | | Ectoderm |
| | Differen- | Mesoderm | Differen- |
| TeSR-E8 | tiation | Differentiation | tiation |
| Control Cells Day 4 | −1.95 | −1.36 | −1.49 |
| Control Cells Day 7 | −1.53 | −0.72 | −1.28 |
| Endoderm Differentiation Day 4 | 0.24 | 0.26 | −1.43 |
| Endoderm Differentiation Day 7 | 1.02 | 0.76 | −0.57 |
| Mesoderm Differentiation Day 4 | −1.25 | 1.59 | −1.12 |
| Mesoderm Differentiation Day 7 | −0.64 | 4.58 | 0.00 |
| Ectoderm Differentiation Day 4 | −1.50 | −1.58 | −0.57 |
| Ectoderm Differentiation Day 7 | −1.21 | −0.11 | 0.64 |

From the results of Tables 25 and 26, it is found that, in “the culture of the target cells” (no differentiation induction stimulation), differentiation toward a specific direction is not observed; in “the culture of the endoderm differentiated cells” (differentiation induction stimulation by Activin-A, Wnt3a and BMP-4), endoderm differentiation proceeds; in “the culture of the mesoderm differentiated cells” (differentiation induction stimulation by BMP-4), mesoderm differentiation proceeds; and in “the culture of the ectoderm differentiated cells” (differentiation induction stimulation by B431542 and Noggin), ectoderm differentiation proceeds.

From these results, it can be said that the evaluation method in which an abundance of a biomarker in a culture supernatant is used as an indicator can reproduce the conventional cell destructive method, and is as excellent method in which change with time of a differentiation state can be evaluated with cells in the same culture dish as targets.

### Data Analysis Based on Ratio of Compounds in Anteroposterior Relation of Metabolic Pathway

For culture supernatants collected from Day 1 to Day 6 of the culture, index values of abundances of substances obtained by LC-MS analysis were calculated. From the index values of kynurenine, tryptophan, ornithine, arginine and putrescine in the culture supernatants of the control cells (undifferentiated maintenance cells), the test cells (endoderm differentiated cells, mesoderm differentiated cells, and ectoderm differentiated cells), a value A/B was calculated which was obtained by dividing an average value B of the index values of the test cells by an average value A of the index values of the control cells.

### Kynurenine/Tryptophan

Tables 27 and 28 show values of A/B of kynurenine as a metabolic product, and values obtained by dividing A/B of kynurenine by A/B of tryptophan which is a precursor of the kynurenine pathway.

TABLE 27
Kynurenine/Tryptophan (mTeSR1)

| | mTeSR1 | | | | | |
|---|---|---|---|---|---|---|
| | Endoderm | | Mesoderm | | Ectoderm | |
| | Differentiation | | Differentiation | | Differentiation | |
| | Kynurenine | Kynurenine/ | Kynurenine | Kynurenine/ | Kynurenine | Kynurenine/ |
| | Alone | Tryptophan | Alone | Tryptophan | Alone | Tryptophan |
| Day 1 | 0.9023 | 0.9163 | 0.9199 | 0.9661 | 0.8529 | 0.9634 |
| Day 2 | 0.4486 | 0.4105 | 0.5372 | 0.5070 | 0.7379 | 0.6674 |
| Day 3 | 0.1889 | 0.1721 | 0.2182 | 0.1799 | 0.7399 | 0.6693 |
| Day 4 | 0.1286 | 0.0475 | 0.1479 | 0.0449 | 0.5916 | 0.2320 |
| Day 5 | 0.1907 | 0.0031 | 0.0984 | 0.0012 | 0.4633 | 0.0093 |
| Day 6 | 0.3526 | 0.0070 | 0.0359 | 0.0003 | 0.3833 | 0.0098 |

TABLE 28
Kynurenine/Tryptophan (TeSR-E8)

| | TeSR-E8 | | | | | |
|---|---|---|---|---|---|---|
| | Endoderm | | Mesoderm | | Ectoderm | |
| | Differentiation | | Differentiation | | Differentiation | |
| | Kynurenine | Kynurenine/ | Kynurenine | Kynurenine/ | Kynurenine | Kynurenine/ |
| | Alone | Tryptophan | Alone | Tryptophan | Alone | Tryptophan |
| Day 1 | 0.9681 | 0.9769 | 0.9745 | 0.9910 | 1.1081 | 1.1096 |
| Day 2 | 0.5220 | 0.5055 | 0.4253 | 0.4203 | 0.6351 | 0.6158 |
| Day 3 | 0.0557 | 0.0542 | 0.1167 | 0.1126 | 0.6368 | 0.6125 |
| Day 4 | 0.0165 | 0.0131 | 0.0306 | 0.0228 | 0.3312 | 0.2538 |
| Day 5 | 0.0126 | 0.0052 | 0.0144 | 0.0064 | 0.1322 | 0.0583 |
| Day 6 | 0.0152 | 0.0049 | 0.0079 | 0.0025 | 0.0677 | 0.0217 |

As shown in Table 4, for kynurenine, due to differentiation induction, an abundance (B) in a culture supernatant of the test cells is decreased relative to an abundance (A) in a culture supernatant of the control cells, and whether or not the test cells are in a differentiated state can be determined even with kynurenine alone (as described above, in the table, a value that deviates more from 1 indicates a larger change from the control cells). On the other hand, as shown in Table 27 (mTeSR1) and Table 28 (TeSR-E8), it is found that, for the ratio of kynurenine to tryptophan, which is the precursor of kynurenine, the difference from the control cells is larger as compared to the case of kynurenine alone, and this tendency becomes more prominent as the number of days of culture increases.

Comparing these results with Tables 25 and 26 which show evaluation results using a cell destructive method, it is found that, in the method of evaluating abundances in a culture supernatant of the test cells, at a stage where the number of elapsed days after the start of the differentiation induction stimulation is small, a significant difference appears between the target cells and the test cells. Further, it is found that, when the ratio of kynurenine to tryptophan rather than kynurenine alone is used as an indicator, the difference from the control cells tends to be more prominent, and thus, the differentiation state of the test cells can be evaluated with higher accuracy.

### Ornithine/Arginine

Tables 29 and 30 show values of A/B of ornithine as a metabolic product, and values obtained by dividing A/B or ornithine by A/B of arginine which is a precursor of the ornithine cycle (urea cycle).

TABLE 29
Ornithine/Arginine (mTeSR1)

| | mTeSR1 | | | | | |
|---|---|---|---|---|---|---|
| | Endoderm | | Mesoderm | | Ectoderm | |
| | Differentiation | | Differentiation | | Differentiation | |
| | Ornithine | Ornithine/ | Ornithine | Ornithine/ | Ornithine | Ornithine/ |
| | Alone | Arginine | Alone | Arginine | Alone | Arginine |
| Day 1 | 0.8824 | 0.8636 | 0.8754 | 0.9015 | 0.8191 | 0.8892 |
| Day 2 | 0.9173 | 0.8697 | 0.7791 | 0.7363 | 1.0158 | 1.0009 |
| Day 3 | 0.8515 | 0.9042 | 0.6125 | 0.5881 | 0.9221 | 1.0511 |
| Day 4 | 0.8213 | 0.6909 | 0.5843 | 0.4243 | 0.9650 | 0.9847 |
| Day 5 | 0.6869 | 0.3975 | 0.5917 | 0.3115 | 0.8816 | 0.6916 |
| Day 6 | 0.6782 | 0.3064 | 0.4975 | 0.1740 | 0.7712 | 0.4924 |

TABLE 30
Ornithine/Arginine (TeSR-E8)

| | TeSR-E8 | | | | | |
|---|---|---|---|---|---|---|
| | Endoderm | | Mesoderm | | Ectoderm | |
| | Differentiation | | Differentiation | | Differentiation | |
| | Ornithine | Ornithine/ | Ornithine | Ornithine/ | Ornithine | Ornithine/ |
| | Alone | Arginine | Alone | Arginine | Alone | Arginine |
| Day 1 | 1.0432 | 1.0727 | 1.0117 | 1.0073 | 1.1216 | 1.0860 |
| Day 2 | 0.8346 | 0.8213 | 0.7999 | 0.7962 | 1.0779 | 1.0942 |
| Day 3 | 0.4761 | 0.4565 | 0.4050 | 0.3849 | 1.0404 | 1.0600 |
| Day 4 | 0.3588 | 0.3070 | 0.1664 | 0.1380 | 0.9389 | 0.9700 |
| Day 5 | 0.3590 | 0.2521 | 0.1297 | 0.0911 | 0.9076 | 0.8407 |
| Day 6 | 0.3042 | 0.2153 | 0.1443 | 0.0952 | 0.9122 | 0.8834 |

As shown in Table 5, for ornithine, due to differentiation induction, an abundance (B) in a culture supernatant of the test cells is decreased relative to an abundance (A) in a culture supernatant of the control cells, and whether or not the test cells are in a differentiated state can be determined even with ornithine alone. On the other hand, as shown in Table 29 (mTeSR1) and Table 30 (TeSR-E8), it is found that, for the endoderm differentiated cells and the mesoderm differentiated cells, for the ratio of ornithine to arginine, which is she precursor of ornithine, the difference from the control cells is larger as compared to the case of ornithine alone, and this tendency becomes more prominent as the number of days of culture increases. From these results, it is found that, by using the ratio of ornithine to arginine rather than ornithine alone as an indicator, the differentiation state of the test cells can be evaluated with higher accuracy.

### Putrescine/Ornithine

Table 31 shows values of A /B of putrescine as a metabolic product, and values obtained by dividing A/B of putrescine by A/B of ornithine which is a precursor of arginine/proline metabolism.

TABLE 31
Putrescine/Ornithine (mTeSR1)

| | mTeSR1 | | | | | |
|---|---|---|---|---|---|---|
| | Endoderm | | Mesoderm | | Ectoderm | |
| | Differentiation | | Differentiation | | Differentiation | |
| | Putrescine | Putrescine/ | Putrescine | Putrescine/ | Putrescine | Putrescine/ |
| | Alone | Ornithine | Alone | Ornithine | Alone | Ornithine |
| Day 1 | 0.9920 | 1.1242 | 0.9331 | 1.0659 | 0.8149 | 0.9948 |
| Day 2 | 1.4154 | 1.5430 | 1.3524 | 1.7357 | 0.9593 | 0.9444 |
| Day 3 | 1.1415 | 1.3406 | 0.9140 | 1.4922 | 1.0112 | 1.0966 |
| Day 4 | 1.3915 | 1.6944 | 0.7268 | 1.2439 | 1.5630 | 1.6197 |
| Day 5 | 4.3553 | 6.3406 | 0.9121 | 1.5415 | 4.6089 | 5.2277 |
| Day 6 | 4.2198 | 6.2219 | 1.1884 | 2.3885 | 4.2743 | 5.5425 |

When an mTeSR1 culture medium is used, for putrescine, due to differentiation induction, an abundance (B) in a culture supernatant of the test cells tends to increase relative to an abundance (A) in a culture supernatant of the control cells. In particular, for the endoderm differentiated cells and the ectoderm differentiated cells, this tendency becomes more prominent as the number of days of culture increases. Therefore, whether or not the test cells are in a differentiated state can be determined with putrescine alone. For the mesendoderm differentiated cells, with putrescine alone, a clear trend was not observed. In contrast, by using the ratio of putrescine to ornithine, which is a precursor of putrescine, the difference from the control cells became larger as the number of days of culture increases, and, also for the mesoderm differentiated cells, whether or not the cells are in a differentiated state can be determined. It is found that, for the endoderm differentiated cells and the ectoderm differentiated cells, the difference from the control cells is larger in the case where the ratio of putrescine to ornithine, which is a precursor of putrescine, is used than in the case where putrescine alone is used, and the differentiation state of the test cells can be evaluated with higher accuracy.

1. A method of evaluating cell differentiation state, comprising:
comparing abundances of indicator substances in a culture supernatant of test cells with abundances of the indicator cells in a culture supernatant of control cells such that a differentiation state of the test cells is evaluated,
wherein the test cells are stem cells for which a differentiation state is unknown or cells differentiation-induced from pluripotent stem cells, the control cells are pluripotent stem cells for which a differentiation state is known, and the indicator substances are a plurality of compounds selected from the group consisting of ornithine, 2-aminoadipic acid, deoxycytidine, tryptophan, alanine, hypoxanthine, uridine, arginine, kynurenine, cystathionine, and putrescine.

2. (canceled)

3. The method according to claim 1, wherein the differentiation state is evaluated based on, for each of the indicator substances, whether or not a ratio of an abundance of an indicator substance in a culture supernatant of the test cells to an abundance of the indicator substance in a culture supernatant of the control cells is equal to or greater than a predetermined threshold or is equal to or less than the threshold.

4. The method according to claim 1, wherein the control cells are cells that are clearly undifferentiated.

5. The method according to claim 1, wherein, the comparing comprises evaluating whether or not the test cells are in a differentiated state or in an undifferentiated state, and evaluating whether a differentiation direction of the test cells that have been evaluated as being in a differentiated state is endoderm, mesoderm, or ectoderm.

6. The method according to claim 5, wherein the comparing comprises analyzing change with culture time of an abundance of an indicator substance in the culture supernatant of the test cells for each of the indicator substances.

7. The method according to claim 5, wherein the comparing comprises subjecting the abundances of the indicator substances to multivariate analysis.

8. The method according to claim 1, wherein the compounds of the indicator substances are selected from the group consisting of kynurenine, 2-aminoadipic acid, ornithine, and deoxycytidine.

9. The method according to claim 1, wherein the compounds of the indicator substances are selected from the group consisting of kynurenine, 2-aminoadipic acid, ornithine, deoxycytidine, tryptophan, cystathionine, hypoxanthine, and uridine.

10. The method according to claim 1, wherein the compounds of the indicator substances are a precursor and a metabolic product in an anteroposterior relation of a metabolic pathway.

11. The method according to claim 10, wherein the precursor is tryptophan, and the metabolic product is kynurenine.

12. The method according to claim 10, wherein the precursor is ornithine, and the metabolic product is putrescine.

13. The method according to claim 10, wherein the precursor is arginine, and the metabolic product is ornithine.

14. The method according to claim 10, wherein the comparing comprises evaluating based on a ratio of an abundance of the precursor to an abundance of the metabolic product in the culture supernatant of the test cells.

15-19. (canceled)

20. A method for culturing cells, comprising:
selecting the test cells based on an evaluation result of the differentiation state of the test cells obtained by the method of claim 1.

21. The method according to claim 1, wherein one of the compounds in the indicator substances is 2-aminoadipic acid.

22. A method of evaluating cell differentiation state, comprising:
comparing an abundance of an indicator substance in a culture supernatant of test cells with an abundance of the indicator substance in a culture supernatant of control cells such that a differentiation state of the test cells is evaluated,
wherein the test cells are stem cells for which a differentiation state is unknown or cells differentiation-induced from pluripotent stem cells, the control cells are pluripotent stem cells for which a differentiation state is known, and the indicator substance is at least one compound selected from the group consuming of ornithine 2-aminoadipic acid, deoxycytidine, tryptophan, alanine, hypoxanthine, uridine and arginine.

23. The method according to claim 22, wherein the comparing comprises evaluating the differentiation state of the test cells based on whether or not a ratio of the abundance of the indicator substance in the culture supernatant of the test cells to the abundance of the indicator substance in the culture supernatant of the control cells is equal to or greater than a predetermined threshold or is equal to or less than the threshold.

24. The method according to claim 23, wherein the control cells are cells that are clearly undifferentiated.

25. The method according to claim 24, wherein the comparing comprises determining that the test cells are in the differentiated state when a ratio of an abundance of at least one compound selected from the group consisting of arginine and tryptophan in the culture supernatant of the test cells to an abundance of the at least one compound in the culture supernatant of the control cells is equal to or greater than a predetermined threshold.

26. The method according to claim 24, wherein the comparing comprises determining that the test cells are in the differentiated state when a ratio of the abundance of at least one compound selected from the group consisting of ornithine and alanine in the culture supernatant of the test cells to the abundance of the at least one compound in the culture supernatant of the control cells is equal to or less than a predetermined threshold.
